(19) United States
(12) Patent Application Publication
(10) Pub. No.: US 2012/0289787 A1
(43) Pub. Date: Nov. 15, 2012

(54) SYSTEM FOR CLINICAL WORKFLOW ENHANCEMENTS USING A BUSINESS RULES ENGINE THAT COLLATES HETEROGENEOUS HEALTHCARE DATA, AND A METHOD THEREOF

(76) Inventors: Michael J. Kurgan, Miami Beach, FL (US); Ranjeet Vijaykumar Deshmukh, Aurangabad Maharashtra (IN)

(21) Appl. No.: 13/107,664

(22) Filed: May 13, 2011

(51) Int. Cl. A61B 5/00

(52) U.S. Cl. 600/300

(57) ABSTRACT

A system for data integration and collation of medical data is described. The system includes a plurality of data sources and a plurality of connectors, wherein the data sources include data from a physiological sensor and a caregiver data store, a connector and source are paired, and each paired connector converts source data between a first format and a second format different from the first format. The system further includes a rules engine and a medical rule associated with a medical protocol and a core transaction processing system configured to apply the medical rule to the data in the second format to implement a compliance rules check of the medical protocol. Also described is a system for monitoring post-procedure complications using a plurality of physiological sensors for collecting medical data; an on-site gateway communicating with the sensors; a server for receiving physiological data recorded by the sensors from the gateway; a database comprising a patient medical record, a prescribed protocol, and a rule set describing the prescribed protocol; and a medical rule evaluator for accessing the rule set and applying the rule set to the physiological data to determine compliance with the prescribed protocol.

### TECHNICAL FIELD

The present teachings are directed toward the improved collection of medical/physiometric instrumentation data. In particular, the disclosure relates to collection of analysis and utilization of medical/physiometric instrumentation data to enhance clinical workflows like monitoring post-procedure complications and preventing fraud.

### BACKGROUND

Medical instrumentation has always produced physiological data. This prior art data collection activity is instrument and processor specific. Moreover, different vendors of the same or similar medical instrumentation use different and varying protocols to collect, store and transmit data. As such, data acquisition, data storage, data retrieval, and data interpretation require custom programming and purchase of vendor specific analysis. Therefore, there is a need to provide a robust across the board analysis tool that works in a heterogeneous environment to collect and collate the data. The collated data can be provided by sensors and other healthcare domain data stores like an Electronic Medical Record (EMR). The collated data can be used to implement clinical workflow enhancements. There is also a need to standardize the heterogeneous data and use of varying analysis.

### SUMMARY

According to various embodiments, a system for monitoring post-procedure complications is described. The system comprises a plurality of physiological sensors for collecting medical data; an on-site gateway communicating with the sensors; a server for receiving physiological data recorded by the sensors from the gateway; a database comprising a patient medical record, a prescribed protocol, and a rule set describing the prescribed protocol; and a medical rule evaluator for accessing the rule set and applying the rule set to the physiological data to determine compliance with the prescribed protocol. In the system, the medical rule evaluator generates an alert if physiological data does not comply with the rule set. The alert can comprise warnings and errors.

In some embodiments, the system can further comprise transmitting the physiological data to a healthcare provider for display. The gateway can comprise a connector to standardize sensor data. The plurality of sensors can be provided by different providers. The sensors can communicate with the on-site gateway using wireless technology. The gateway can communicate with the server using one or more of a leased line, telephone, cellular service, fiber optics, or Internet. The gateway can be disposed remotely from the healthcare provider.

In some embodiments, a payment authorization subsystem can be activated when the medical rule evaluator verifies delivery of medical procedure per the prescribed protocol. The gateway can tag the data with a patient ID and a patient location. The prescribed protocol can comprise a post-procedure complication monitoring protocol.

According to various embodiments, a system for fraud prevention in delivery of home healthcare services is described. The system comprises: a medical data entry device adapted to receive a service provider ID and a service provided ID; a plurality of physiological sensors for monitoring a patient; and a gateway to determine a patient ID, a patient's location, and provide the sensed medical data to the medical data entry device.

In some embodiments, the physiological data comprises one or more of a patient's weight, oxygen level, blood pressure, glucose level, heart rate or a combination thereof. The system can further comprise a server to receive the service provider ID, the service provided ID, the patient ID, the patient location, and the sensed medical data. The server can evaluate the service provided ID to locate an associated rule set and verifies delivery of the service by applying the associated rule set to the physiological medical data. In some embodiments, the server authorizes payment to the service provider ID after verifying delivery. Sometimes, the server can verify that the patient's location is associated with a set of locations where service to the patient can be provided. The gateway can include a patient ID scanner. The gateway can include a GPS sensor. The gateway can be disposed in the medical data entry device.

According to various embodiments, a system for data integration and collation is described. The system comprises a data collection system comprising a plurality of data sources and a plurality of connectors, wherein the data sources include data from a physiological sensor and a caregiver data store, a connector and source are paired, and each paired connector converts source data between a first format and a second format different from the first format. The system further comprises a rules engine and a medical rule associated with a medical protocol; and a core transaction processing system configured to apply the medical rule to the data in the second format to implement a compliance rules check of the medical protocol.

In some embodiments, the application of the medical rule confirms patient compliance with the medical protocol. In some embodiments, the application of the medical rule confirms caregiver compliance with the medical protocol. In other embodiments, the application of the medical rule generates health status data.

According to various embodiments, the health status data is used for real-time monitoring of the patient. The application of the medical rule can generate a mental health profile of the patient. The data sources can comprise one or more of a body area network sensor, an Electronic Health Record (EHR), an EMR, an Hospital Information Services (HIS) record, a public personal profile, a restricted personal profile, geo-location data, or route data.

The connector converts data from the second format to the first format and the system generates an external actionable event. The system can further comprise a data integrator to collate data from at least two data sources.

### BRIEF DESCRIPTION OF THE DRAWINGS

The same reference number represents the same element on all drawings. It should be noted that the drawings are not necessarily to scale. The foregoing and other objects, aspects, and advantages are better understood from the following detailed description of a preferred embodiment of the invention with reference to the drawings, in which:

FIG. 1 is an embodiment of a system 100 used by medical facilities;

FIG. 2 is an embodiment of how software can be logically connected to provide the systems and methods of the present teachings;

FIG. 3 is a logical diagram of providing business services according to one embodiment;

FIG. 4 is an embodiment of a data integrator according to one embodiment;

FIG. 5 is an embodiment of a data flow diagram for a use case or workflow usable with diabetic patients;

FIG. 6 is an embodiment of a data flow diagram for a use case or workflow usable for fraud detection;

FIG. 7 is an embodiment of a data flow diagram for a use case or workflow usable for monitoring post-procedure monitoring of patients who have suffered from congestive heart failure;

FIG. 8 is an embodiment of a data flow diagram for a use case or workflow usable for mental health profile creating and scoring; and

FIG. 9 is an embodiment of a data flow diagram for a use case or workflow usable for care protocol compliance monitoring.

### DETAILED DESCRIPTION

The teachings herein are directed to a system that accepts sensory input from non-homogenous sensors and/or non-homogenous healthcare domain data stores. The system presents the data in a homogenous user interface as needed by a healthcare provider. In some embodiments, the healthcare domain data stores can include such exemplary systems as an EMR system holding patient data, an ERP system or other data store systems located at point of care. The collected data can be converted and formatted, in order to obtain homogenized data. The homogenized data from the various sources can be collated. As such, the collated data can be provided by sensors and healthcare domain data stores. In some embodiments, the collated data can be used implement clinical workflow enhancements.

The system provides a user interface that can be used by a healthcare provider. In some embodiments, the user interface can be used to make determinations about protocol compliance by the medical professional. In some embodiments, the system can access a patient's medical record from a healthcare domain data store and determine if the sensed data comports with the protocol prescribed to a patient. Warnings and errors for failure to comply with the protocol can be generated. The data can be collected at a patient's home, at a medical facility or both. The sensors can communicate with an on-site gateway, e.g., over Bluetooth, and the data recorded by the sensors can be forwarded to a central server, e.g., over the Internet. In some embodiments, a system operates with non-homogenous sensors (sensors made by different manufacturers).

The present teachings save costs in custom programming because ‘connectors’ for instrument/protocol specific data acquisition are provided. The connectors can be built into the system. In some embodiments, the connectors can be provided to a user on Software as a Service (SaaS) basis. So for example, a newly supported device/instrument can be available for all users without significant re-engineering costs to the connector provider. In some embodiments, the connectors can be borrowed or purchased from industry approved solutions in market. In some embodiments, the connectors can be indigenous.

In a preferred embodiment, a data gateway capable of using configured connectors to accept data from various sources is described. The gateway can be connected to data producers or sources, which can be humans or physiometric instruments. Connectors can be used to connect to data sources.

The gateway can be connected to data sinks or consumers, which can be humans or physiometric instruments. Data consumers generally interpret the data. Connectors can be used to connect to data consumers.

The correlation of the acquired data is enhanced after it has been homogenized/standardized within a core system. In some embodiments, the interpretation of data, either by humans or processors is targeted to specific use cases. In specific use cases, meaningful correlation of such homogenized data greatly enhances the value of the data. The correlation can be enhanced by the following exemplary means:
Increase the scope of relations from targeted use case or workflow to hospital/enterprise wide scope
Expose homogenized data for writing enterprise level business rules while keeping device dependencies transparent to end user
Make correlated data available for real-time as well as analyzed decisions (this is not the case with currently available data interpreters)
Allow access to singular (i.e., current heart rate) as well as historical (average sugar level in last 3 months) data parameters while creating business rules.
Provide out of the box business rules (i.e., never events) which can be readily adapted by enterprises using our system.

The standardized data can be plugged into a medical protocol. In some embodiments, the standardized data can be integrated and/or associated with a Patient's Medical Record (PMR). In some embodiments, the standardized data can be relayed back to any EMR system connected to the system. The integration can be used to provide significant benefits to a user. For example, the integration can post alarms if recommended protocols are not followed by a patient or a service provider. In some embodiments, payments to a service provider can be authorized post acquisition and integration of data related to the service provided. In some embodiments, the integration can be used for findings of fact or for other data mining activities.

The present teachings use business rules at enterprise level rather the traditional use of triggers at a workflow level. For example, the system can be used to enhance quality of care and minimize human errors. A business rule that ensures that a specific drug must be dispatched by inventory for given treatment, or it can ensure that the correct patient is being given the treatment or surgery \at the enterprise level can be modeled as:

When:
(Any patient with SW financial rating <X) AND (scheduled for a surgery costing >Y) AND with past non-pay cases==TRUE)

Then:
The patient should be alarmed for.
   Another example assuring quality of care is:

When:
(A patient scheduled for surgery X has arrived into operation room Y)

Then:
(There should have a recent entry in inventory system for dispatch of drug Z, which is required for surgery X).

Presently, there is a push in the medical care industry to switch from pay per procedure to pay per bundle by an insurance carrier. The care giver is paid a fixed price for services to be rendered as a bundle rather than individually, i.e., the care giver is paid once. Examples of such treatment include treating for Congestive Heart Failure (CHF) (heart attack) including in hospital and home care post procedure. In the pay per bundle, a care provider can increase profit and reduce risk by (1) avoiding duplication of procedures by various specialists needed for treatment and (2) by verifying that post-procedure protocols that reduce complications are followed in the hospital and at home.

For example, for a CHF patient weight change within 60 days of a heart attack can indicate problems. In another example, when a bulimic or anorexic patient induces vomiting, his heart stops—this can be seen by a blood pressure sensor. In another example, a patient's insulin levels retrieved from the glucose monitor can indicate when an insulin shot was received by a diabetic patient.

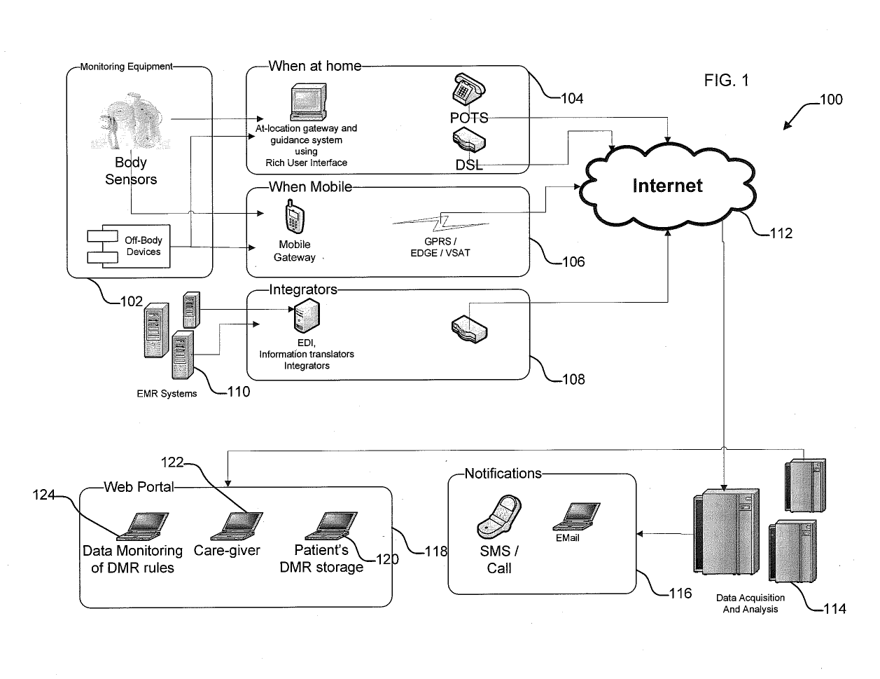

FIG. 1 is an embodiment of a system 100 used by medical facilities. At the technical core, system 100 integrates healthcare related heterogeneous data from various sources. Data is collected from monitoring equipment 102 including on-body or off-body physiological sensors. Monitoring equipment 102 can transmit physiological data to a gateway 104, for example, when the patient is at home or a medical facility. In some embodiments, monitoring equipment 102 can transmit physiological data via a cellular phone 106. Data from monitoring equipment 102 or from cellular phone 104 can be transmitted over a network 112 such as the Internet to a server 114. In some embodiments, an integrator 108 can interface with EMR systems 110. Data from integrator 108 can be transmitted over network 112 to server 114. Integrator 108 can connect to multiple EMR systems. Server 114 can provide data acquisition and analysis. Server 114 can also comprise a rule database. System 100 can be used for specific workflows or use cases of the same to generate actionable events for several applications including medical compliance check, patient monitoring, caregiver/patient alerts and service validation. System 100 can provide alerts using a notification system 116. System 100 can present the collected and analyzed data using a presentation system 118. Presentation system 118 can comprise, for example, a web-server.

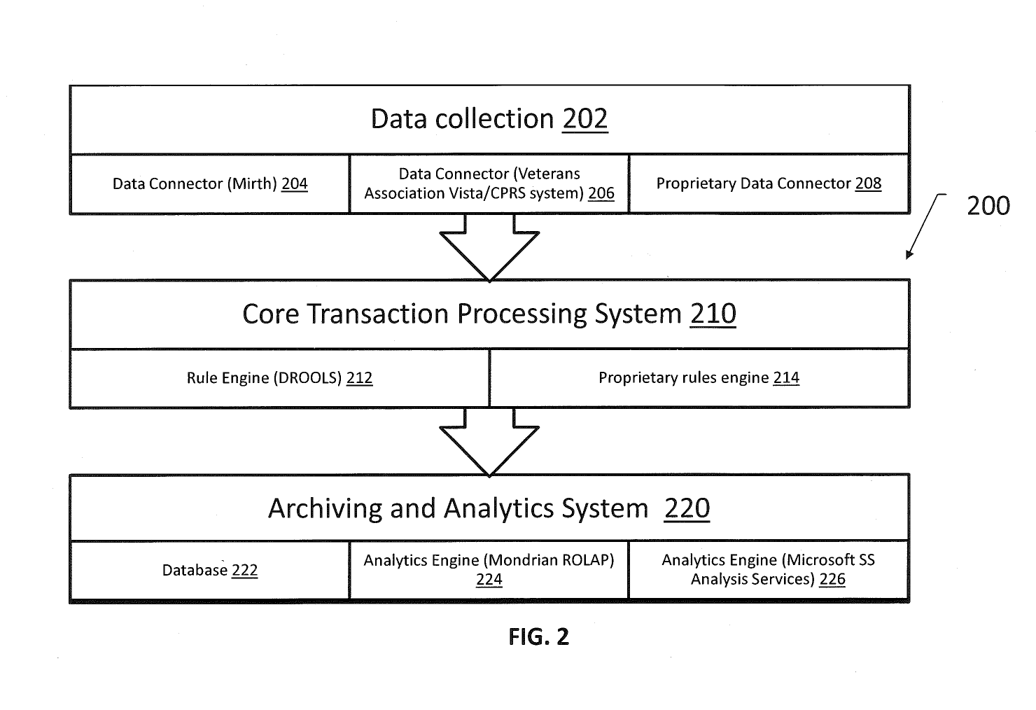

FIG. 2 is an embodiment of how software can be logically connected to provide the systems and methods of the present teachings. A technology stack 200 can be used to implement the software. In a preferred embodiment, data collection 202 can be provided using a combination of various data connectors. In some embodiments data connector 204 can be provided by Mirth connect software described at http://www.mirthcorp.com/products/mirth-connect. In some embodiments, a data connector 206 for connecting a data flow to and from U.S. Veterans Association (VA) EMR system can be provided. Other connectors 208 can be provided as needed. The data collected via data connectors 202 is then homogenized and processed through a core transaction processing engine 210. Transaction processing engine 210 can use active and passive alerts. In some embodiments, the alerts can be via Rule engine 212 implemented using DROOLS (a Java based business logic processor). In some embodiments Rule engine 212 can be used in used in conjunction or replaced with a propriety rules engine 214. Propriety rules engine 214 can be written in Microsoft VC++ and .Net. Data from data connectors 202 can be processed real-time for customer defined business rules resulting into actionable events.

Processed data is then stored using an archival and analytics system 220. In some embodiments, archival system 220 can include a database 222, for example, a MySQL database. Archival and analytics system 220 can be exposed to customers for reporting. Archival and analytics system 220 can comprise an analytics engine 224 that uses, for example, an open source Mondrian ROLAP interface. Archival and analytics system 220 can comprise an analytics engine 226 that uses, for example, Microsoft SSAS (SQL Server Analytical Services) 224.

### Application of Technology

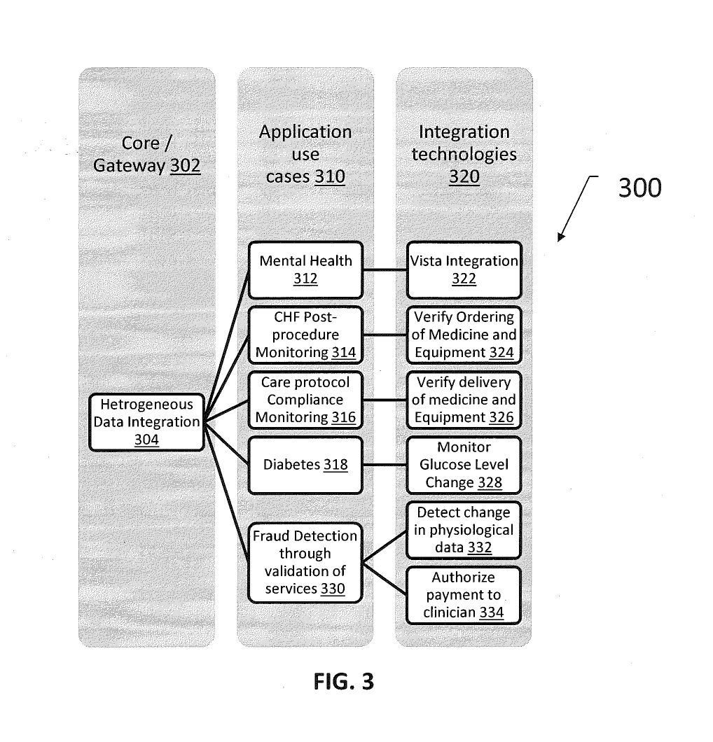

FIG. 3 is a logical diagram of how the teachings herein can be utilized to provide business services. The core/gateway 302 technology stack described above is further utilized for various business and domain specific applications. Applications 310 use the core/gateway 302 and are viewed as ‘use cases’. The applications 304 can utilize integration technologies 320 to provide desired business solutions. The proposed architecture of core/gateway technology 302 includes one or more of:
ESB (Enterprise Service Bus), e.g., Microsoft BizTalk server
DSS (Decision Support Systems) and Clinical DSS
Business Rules Management Systems (BRMS) e.g., IBM Websphere ILOG BRMS
Core: ‘pre-programmed’ business rules to be made available to customer in SAAS (Software as a Service) model. Logical groups of these pre-programmed business rules form a ‘use case’ supported. The pre-programmed rules can also be viewed as standard business practices by smaller customers.
A Business Rules Processing layer for Healthcare to alter performance of the software according to use case or workflow in either real-time, offline, or batch mode.

Core/gateway 302 can begin by standardizing data from non-homogenous physiological sensors and perform heterogeneous data integration 304. Data from heterogeneous data integration can be used by various use case or workflow applications 310 such as mental health use case 312, a CHF Post-procedure Monitoring use case 314, a Care protocol Compliance Monitoring use case 316, a diabetes use case 318, and a fraud detection through validation of services use case 330.

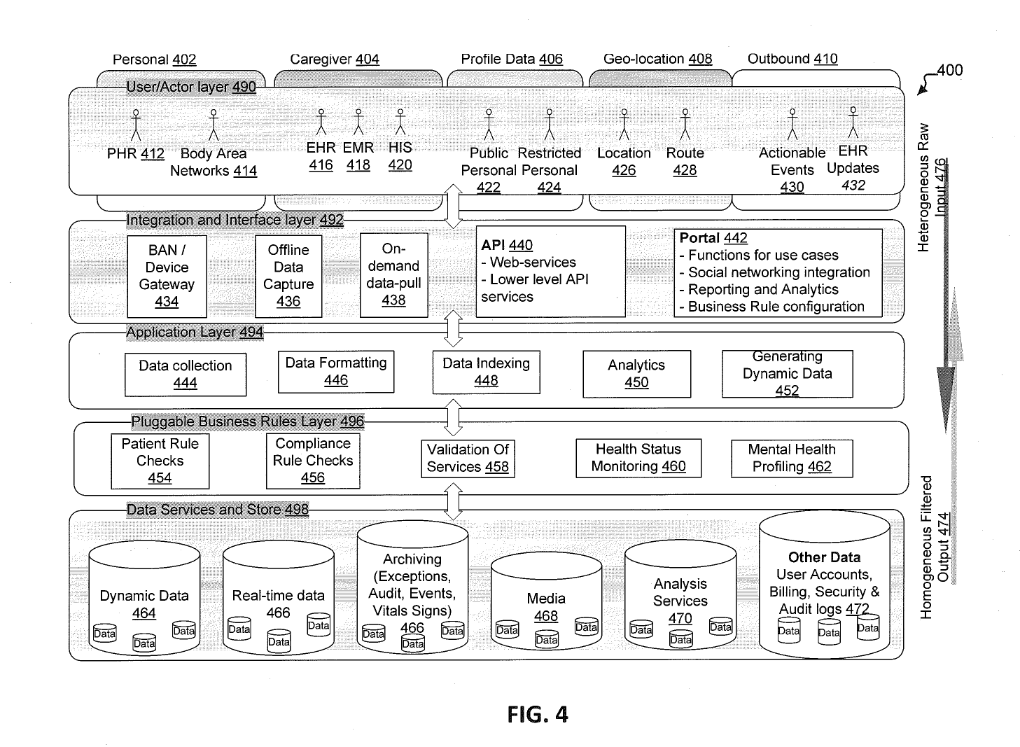

FIG. 4 is an embodiment of a data integrator 400 that accepts data from various heterogeneous health, medical and bio data sources, stores the data in a homogenous format, collates the data and makes the data available for other applications for applying business rules. A user/actor layer 490 of data integrator 400 can obtain personal data 402, caregiver data 404, profile data 406, and/or geo-location data 408. The data can be divided into one or more of these categories. Data integrator 400 can retrieve a Personal Health Record (PHR) 412, physiological data collected from a body area network (BAN) 414, an EHR 416, an EMR 418, data from a HIS 420, or a combination thereof. In some embodiments, profile data 406 like a public personal profile 422 or a restricted personal profile 424. In some embodiments, geo-location data 408 can comprise a location 426 or a route 428. In some embodiments, data integrator 400 can include outbound connectors 410 for actionable events 430 or for EHR updates 432. User/actor layer 490 can interface with an integration and interface layer 492 which can include connectors to the various categories of data available to user/actor layer 490. In some embodiments, one or more of a BAN/device connector, an offline data capture 436, or an on-demand data-pull 438 can be provided. Integration and interface layer 492 can also provide an application protocol interface (API) 440. API 440 can provide web-services. In some embodiments, API 440 can provide lower level API services. In other embodiments, integration and interface layer can include a portal 442. Portal 442 can provide functions to access, manage and define use cases. In some embodiments, portal 442 can provide an interface for social networking integration. In other embodiments, portal 442 can provide an interface for reporting and analytics. In a preferred embodiment, portal 442 can include a business tool configuration and definition interface.

Integration and interface layer 492 can interface with an application layer 494. Application layer 494 can include one or more of a data collection 444, data formatting 446, data indexing 448, analytics 450, and generating dynamic data 452 module. Application layer 494 can interface with a pluggable business rules layer 496. Pluggable business rules layer 496 can include one or more of a patient rules check 454, a compliance rules check 456, a validation of services 458, a health status monitoring 460, a mental health profiling 462 module. Pluggable business rules layer 496 can interface with a data services and store layer 498. Data services and store layer 498 can be capable of storing dynamic data 464, real-time data 466, archiving 468, media 468, analysis services 470, or any other data 472 needed by the data integrator 400. Archiving 468 can store exceptions, audit events, events, vital signs etc. Other data 472 can include, for example, user accounts, billing, security logs, or audit logs. In some embodiments, application layer 494 can collate the data received at user/actor layer 490. According to various embodiments, pluggable business rules layer 496 can collate the data received at user/actor layer 490.

The following examples illustrate how the present teachings can be used.

### Diabetes

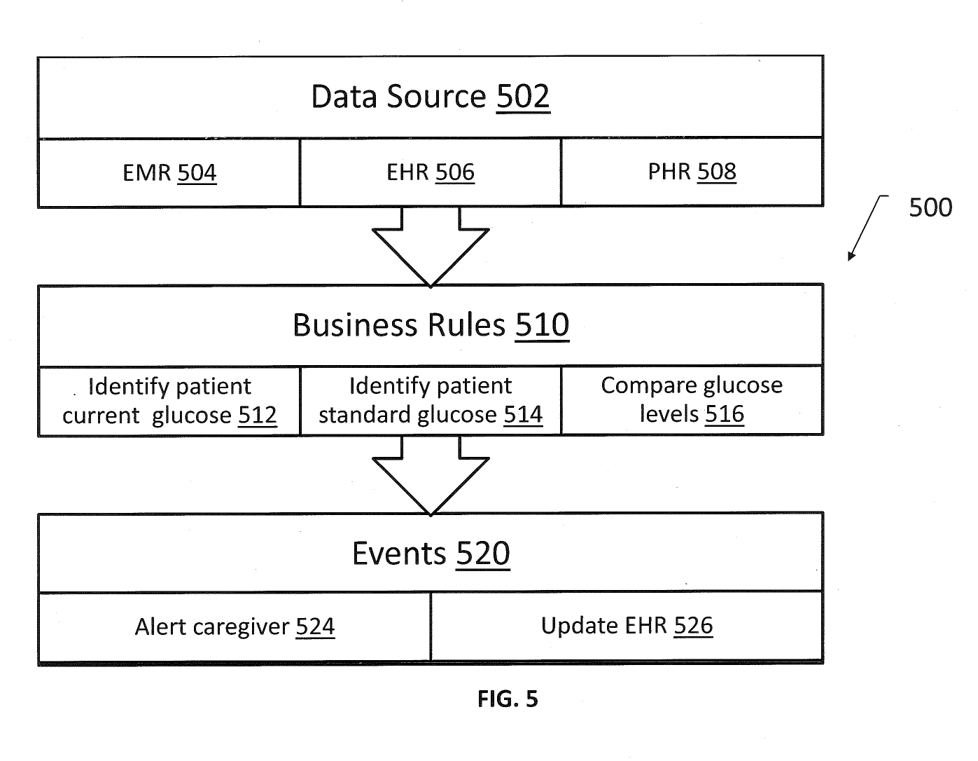

FIG. 5 illustrates a data flow diagram for a use case or workflow 500 usable with diabetic patients. Using data 502 obtained from multiple healthcare related systems like EMR 504, EHR 506 or PHR 508 a system can generate actionable events 520 for diabetic patients with or without co-morbidities. By applying business rules 510, data from EMR 504, EHR 506 or PHR 508 can be collated. When a patient has a higher than usual glucose level norm per rules 512, 514, the two levels can be compared per rule 516. As such, when a patient gets admitted in a hospital, the patient's medical history can be provided to use case 500. If business rules 510 generate an event to notify a caregiver 524 about the expected higher glucose level and a suggestion to take care of the glucose level before the step of getting the glucose level down is suggested. Additionally, business rules 510 can update EHR 526.

When—
Patient.EMR.standardGlucodeLevel>Standards.glucoseLevel OR
Patient.EHR.standardGlucodeLevel>Standards.glucoseLevel OR
Patient.PHR.standardGlucodeLevel>Standards.glucoseLevel

Then—
Patient.Notifications.Alert (“Watch for abnormal glucose level”, Priority.high).

### Fraud Detection

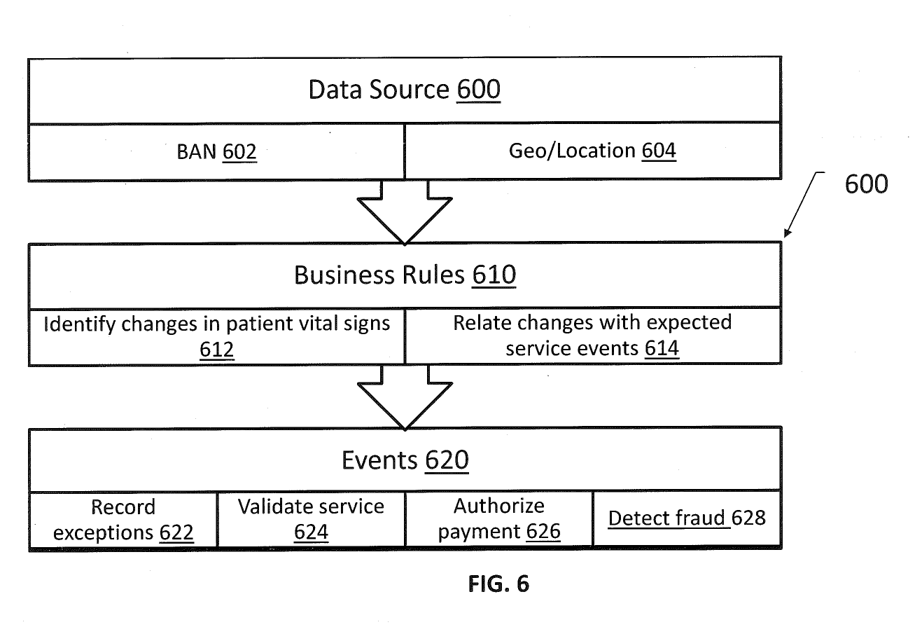

FIG. 6 is an embodiment of a data flow diagram for a use case or workflow 600 usable for fraud detection. Using data 602 obtained from a sensor on a body area network 604 and a Geo-location sensor 606, use case 600 can validate delivery of health services. When a caregiver provides a patient a pre-scheduled service which has recordable impact on body vitals, business rules 610 can collate the data from body area network 604 and Geo-location sensor 606. The collated data can identify physiological changes in a patient's vital signs 612. The physiological data can be related to changes with expected service events 614. The results from the changes with expected service events 614 can generate corresponding events 620. Events 620 can record exceptions 622, validate service 624, authorize payment 626 or detect fraud 628.

When—
Organization.Caregiver.ScheduledPatientVisit.Time <is in> +/−2 hours AND
Organization.Caregiver.Location <is NOT in proximity of> Organization.Caregiver.ScheduledPatientVisit.Patient.location OR
Organization.Caregiver.ScheduledPatientVisit.Patient.VitalSigns <Do not show> Organization.Caregiver.ScheduledPatientVisit.Treatment.ExpectedResults

Then—
Organization.Notifications.Alert (“Caregiver might have missed the scheduled appointment”, Priority.high).

### Congestive Heart Failure—Post Procedure Monitoring

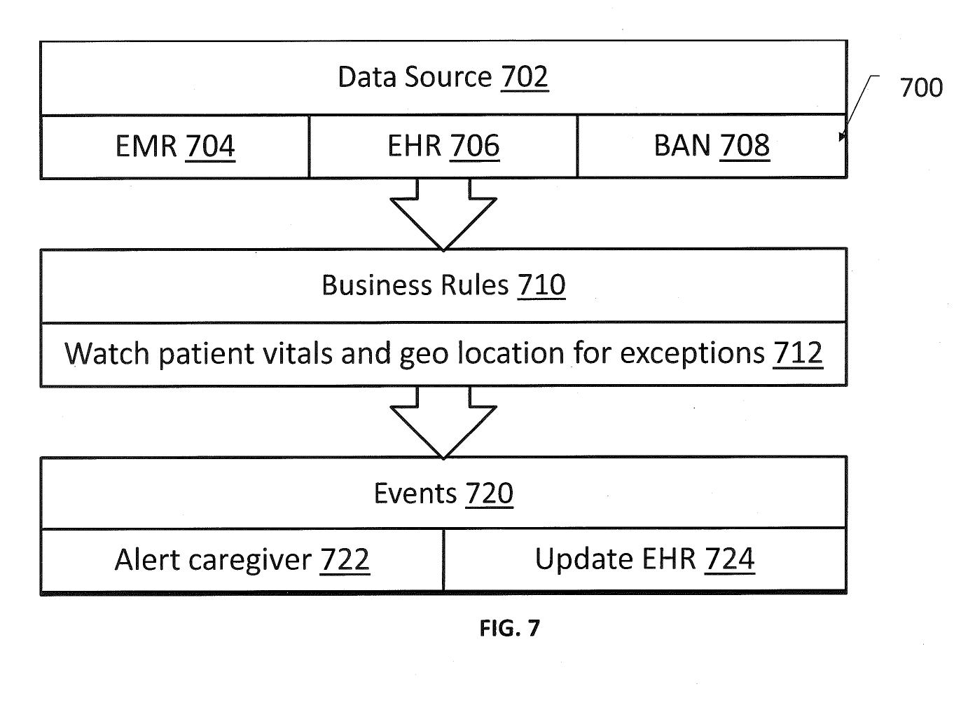

FIG. 7 is an embodiment of a data flow diagram for a use case or workflow 700 usable for monitoring post-procedure monitoring of patients who have suffered from congestive heart failure. Use case 700 can be used for remote monitoring of discharged patients. When, a patient is discharged from hospital after a congestive heart failure treatment, the patient needs to be monitored real time. Using data 702 obtained from multiple healthcare related systems like EMR 704, EHR 706 or BAN sensors 708 and applying business rules 710, use case 700 can generate actionable events 720 for the treatment of the patient. In some embodiments, by applying business rules 710, data from EMR 704, EHR 706 or BAN sensors 708 can be collated. In some embodiments, BAN sensors 708 can comprise body area networked devices. BAN sensors 708 can monitor the patient while he is stationary—for example, at home—or when the patient has restricted mobility. Business rules 710 can monitor equipment and services and can generate events 720. Events 720 can be to alert caregiver 722. The alert can raise alarms/notifications to the caregiver if something goes ‘wrong’, as defined by business rules 710. Additionally, business rules 710 can update EHR 726 with the progress of the patient. For example, the rule structure can comprise:

When—
Patient.RealtimeData.HeartRate>Standards.HeartRate AND
Patient.EHR.UnderCHFMonitoring==True

Then—
Patient.Caregiver.Notifications.Alert (“Heart rate out of bounds”, Priority.high).

### Mental Health

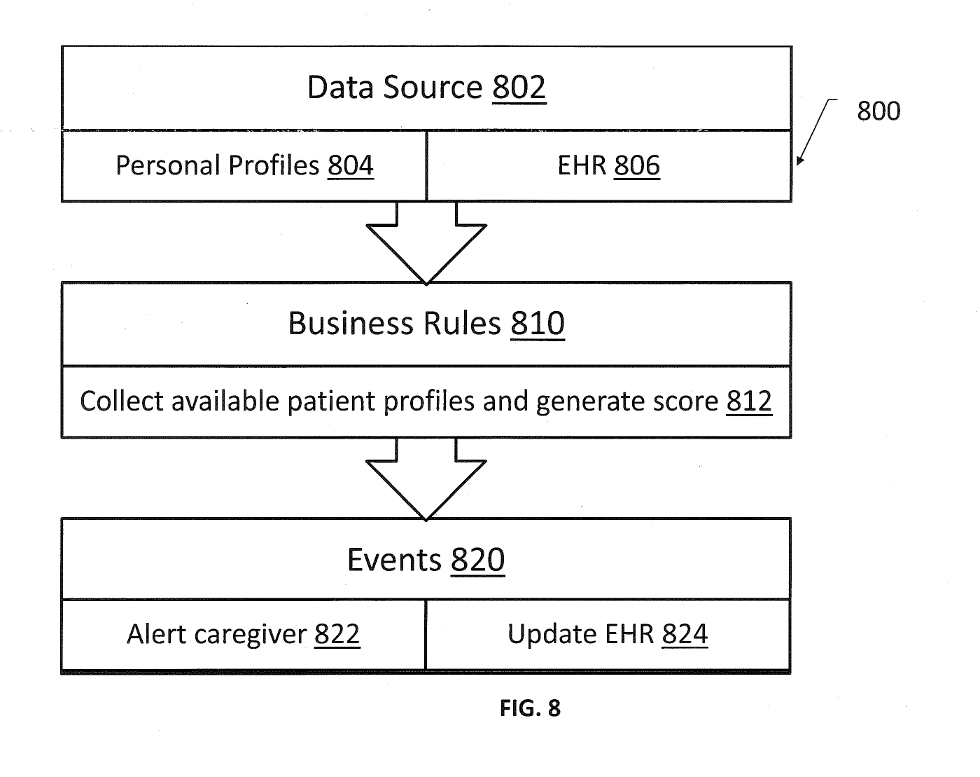

FIG. 8 shows an embodiment, where a system 800 can be used to create and score individuals profile using data obtained from an individual EHR. The information can include public and restricted personal profiles. For example, data source 802 can comprise a patient intake system. When a new EHR 806, for example, a veteran's record, is received into the EHR system, the system can invoke business rule 810. Business rule 810 can retrieve a profile 804 and EHR 806. In some embodiments, profile data 804 may not exist in the healthcare data domain. In such events, profile data 804 can be collected by business rule 810 from government sources (e.g., military records, NCIC database), or from private sources (e.g., credit reports, employment records), or from other public sources (e.g., facebook, linkedin) to generate one or more profiles. This data can be collated. Per the rule engine 810, the patient's mental health is then assessed by the system or by a health care provider and a score is generated. If deemed medically necessary by the system and/or health care provider, events 820 can be generated.

Events 820 can alert caregiver 822 to contact the patient. In some embodiments, alert caregiver 822 can contact the patient through the web for a mental health assessment built for suicide prevention. The resulting scores of the assessment packet from business rule 810 can then be entered into the EHR system for clinicians review. For example, the rule structure can comprise:

When

Patient.PerformMentalHealthCheck==true AND

Patient.MentalHealthAssesmentDetails <are> available

Then—

NewScore=Patient.MentalHealthAssesmentScore+

Patient.MentalHealthAssesmentDetails+

Patient.PublicFeeds.FinancialScore+

Patient.PublicFeeds.LegalScore

Patient.EMR.SubmitClinicalNote(NewScore).

### Care Protocol Compliance Monitoring

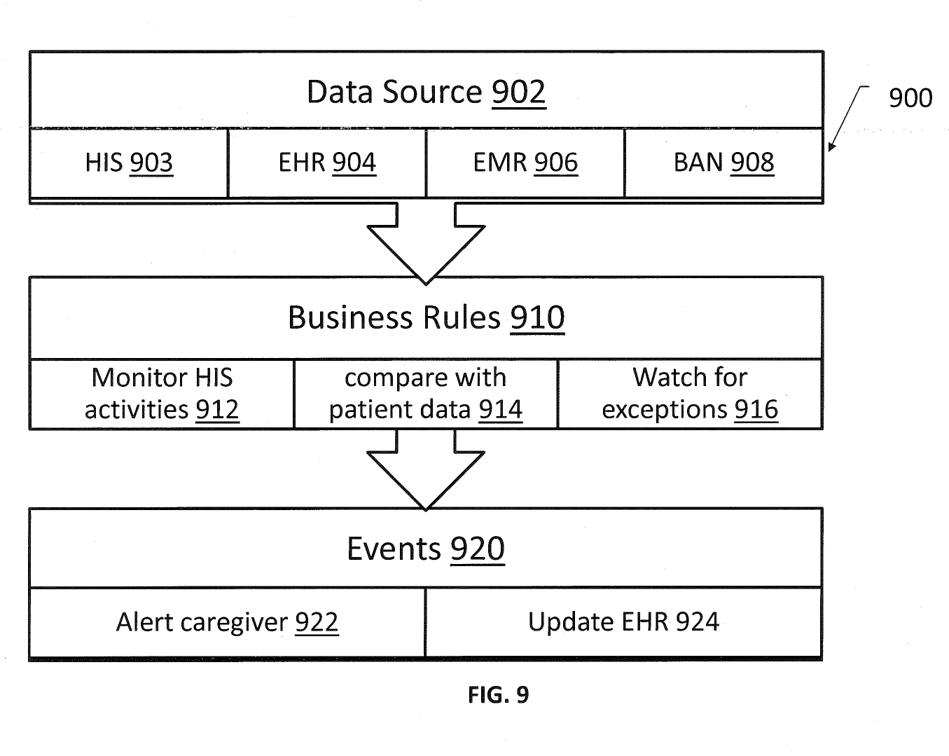

FIG. 9 an embodiment of a data flow diagram for a use case or workflow 900 to validate compliance against configurable protocols and care workflows. Using data 902 obtained from multiple healthcare related systems like HIS 903, EHR 904, EMR 906 or BAN sensors 908 and applying business rules 910, use case 900 can generate actionable events 920 for the treatment of the patient. In some embodiments, by applying business rules 910, data from HIS 903, EHR 904, EMR 906 or BAN sensors 908 can be collated. Business rules 910 in the target hospital can monitor activity of HIS 912 within the hospital. Business rules 910 can then compare the activity with patient data 914 and watch for exceptions 916. In this manner, business rules can evaluate whether a protocol is being violated. For example, if a patient is undergoing certain operative procedure, business rules 910 can check if the expected set of medications is dispatched from inventory. Another example would be to monitor certain ‘never events’ and prevent them from occurring. Business rules 910 can monitor equipment and services and can generate events 920. Events 920 can be to alert caregiver 922. The alert can raise alarms/notifications to the caregiver if something goes ‘wrong’, as defined by business rules 910. Additionally, business rules 910 can update EHR 924 with the progress of the patient. For example, the rule structure can comprise:

When—
Patient.RealtimeData <indicates> Standards.NeverEvents OR
Patient.EMR <indicates> Standards.NeverEvents OR
Patient.EHR <indicates> Standards.NeverEvents OR
Organization.HIS.Events <indicates> Standards.NeverEvents

Then—
Patient.Caregiver.Notifications.Alert (“Patient candidate for Never event <event description>”, Priority.high).

### Integration Process an Exemplary RPC Object Model for Vista

‘Vista’ is a historical EHR/EMR system being used by certain U.S. hospitals for a long time. The system is adapted and customized by certain government agencies such as VA and Department of Defense (DoD) for storing medical records of individuals. The system is not so open for third party developers outside of VA and DoD, and currently there is a lack of publicly available tools to integrate with Vista. Current challenges can be summarized as:
Tools available to integrate with Vista provide high level support for HL7 or RPC calls. However there is no specific support to perform certain system functions.
Vista implementations may differ across locations of VA hospitals and there is no publicly available common API to provide integration.
Vista accepts ‘CPRS’ as its client software. CPRS talks with Vista using ‘RPC calls’ these RPC calls are very specific and may differ across locations/Vista installations. There is not a publicly available authorized list of RPC calls for outside integrators.

An integration package can be trained with an instance of Vista server and the trained system can later be utilized for performing specific integration tasks. The integration package has following parts:
A ‘recorder’ module which listens and records RPC traffic between CPRS and Vista in a log file
A ‘player’ module which replays the traffic recorded in log file in earlier step
An object based substitution engine written in Java which substitutes key data elements of the RPC calls being replayed thus allowing external software to repetitively replay log files with different data elements to perform the job frequently without involving manual usage of CPRS.

The use case or workflow which needs to be automated for integration can be identified. Examples of a workflow or use case include:
a. Add/view a patient record
b. Add/view a clinical note for a patient
c. Add clinical reminder into clinical note for given patient, such as, AUDIT-C, PHQ-9, TBI, MST, Tobacco, PC_PTSD
d. Retrieve list of registered patients to check if a patient already has a record in Vista
e. Digitally sign or not sign a newly created/existing note. Document in the clinical note a need for placing a consult to other departments.

The system can perform the use case or workflow through CPRS and record the RPC calls into a log file. Then the ‘player’ code is changed to set values to be substituted in the RPC calls being replaced. Finally, the system replays the log files with different values to perform the same use case in a black box mode.

For adding a clinical reminder into an existing patient record in Vista. The EMR records maintained within Vista are not publicly accessible. Information provided by VA on the RPC calls is described, for example, at: http://www.va.gov/vdl/documents/Infrastructure/Remote_Proc_Call_Broker_(RPC)/xwb1_{—}1p47r elease_notes.doc. The following RPC commands will be updated real time using value substitution as described:

XUS AV CODE

ORWU DT5001

TMG CONSOLE SERVER QUERY

TMG CONSOLE SHUT DOWN

TIU CREATE RECORD

TIU LOCK RECORD

ORWPCE SAVE

TIU UPDATE RECORD

TIU SET DOCUMENT TEXT

TMG CONSOLE INITIATE

ORQQVI NOTEVIT

TIU UNLOCK RECORD

ORWDX UNLOCK

ORWOR UNSIGN

TIU SIGN RECORD

ORWPT LASTS

Following are the assumed interpretations of the data source to be consumed by the system described. An EMR comprises medical information of a person stored at unit level caregiver, mostly about current instance of care. An EHR comprises medical and health related information of a personal stored at global level of the caregiver, mostly about historical records. A Personal Health Record (PHR) comprises health records in possession of an, individual. A Body Area Network (BAN) comprises data about vital signs collected from devices worn on or in proximity of body. A HIS can comprise software systems running in a hospital, including but not limited to, Patient Intake, Operations Room Mgmt, ERP, Inventory, Billing, etc.

In some embodiments, personal profiles are publicly available and legally obtainable. In some embodiments, private profile data comprises profiles including but not limited to social network profiles, financial data, driving records, legal records.

In some embodiments, dynamic dimensional data is not restricted to a business of medical function or use case. This is a homogeneous form of data obtained from heterogeneous data sources.

Actionable Events comprise events, alerts and notifications generated by the system when the data qualifies configurable business rules.

Business Rules comprise Business function/domain/use case specific rules which are user configured, and the system needs to apply on available data.

The EHR server software used and maintained by certain U.S. government agencies including VA and DoD is named Vista. CPRS is the EHR client software used and maintained by certain U.S. government agencies including VA and DoD. RPC is the TCP/IP based non-public interface CPRS uses to talk with Vista.

The various embodiments described above are provided by way of illustration only and should not be constructed to limit the invention. Those skilled in the art will readily recognize the various modifications and changes which may be made to the present invention without strictly following the exemplary embodiments illustrated and described herein, and without departing from the true spirit and scope of the present invention, which is set forth in the following claims.

We claim:

1. A system comprising:
a plurality of physiological sensors for collecting medical data;
an on-site gateway communicating with the sensors;
a server for receiving physiological data recorded by the sensors from the gateway;
a database comprising a patient medical record, a prescribed protocol, and a rule set describing the prescribed protocol; and
a medical rule evaluator for accessing the rule set and applying the rule set to the physiological data to determine compliance with the prescribed protocol;
wherein the medical rule evaluator generates an alert if physiological data does not comply with the rule set.

2. The system of claim 1, wherein the alert comprises warnings and errors.

3. The system of claim 1, further comprising transmitting the physiological data to a healthcare provider for display.

4. The system of claim 1, wherein the gateway comprises a connector to standardize sensor data.

5. The system of claim 1, wherein the plurality of sensors are provided by different providers.

6. The system of claim 1, wherein the sensors communicate with the on-site gateway using wireless technology.

7. The system of claim 1, wherein the gateway communicates with the server using one or more of a leased line, telephone, cellular service, fiber optics, or Internet.

8. The system of claim 1, wherein the gateway is disposed remote from the healthcare provider.

9. The system of claim 1, further comprising a payment authorization subsystem activated when the medical rule evaluator verifies delivery of medical procedure per the prescribed protocol.

10. The system of claim 1, wherein the gateway tags the data with a patient ID and a patient location.

11. The system of claim 1, wherein the prescribed protocol comprises one or more of a quality of service protocol, a mental health check protocol, a post procedure complication monitoring protocol, a diabetes monitoring protocol, a vital sign monitoring protocol, a fraud prevention protocol, a delivery of prescribed services protocol, or a combination thereof.

12. A system and method for fraud prevention in delivery of home healthcare services:
a medical data entry device adapted to receive a service provider ID and a service provided ID;
a plurality of physiological sensors for monitoring a patient; and
a gateway to determine a patient ID, a patient's location, and provide the sensed medical data to the medical data entry device.

13. The system of claim 12, wherein the physiological data comprises one or more a patient's weight, oxygen level, blood pressure, glucose level, heart rate or a combination thereof.

14. The system of claim 12, further comprising a server to receive the service provider ID, the service provided ID, the patient ID, the patient location, and the sensed medical data.

15. The system of claim 14, wherein the server evaluates the service provided ID to locate an associated rule set and verifies delivery of the service by applying the associated rule set to the physiological medical data.

16. The system of claim 15, wherein the server authorizes payment to the service provider id after verifying delivery.

17. The system of claim 15, wherein the server verifies that the patient's location is associated with a set of locations where service to the patient can be provided.

18. The system of claim 12, wherein the gateway includes a patient ID scanner.

19. The system of claim 12, wherein the gateway includes a GPS sensor.

20. The system of claim 12, wherein the gateway is disposed in the medical data entry device.

21. A system comprising:
a data collection system comprising a plurality of data sources and a plurality of connectors, wherein the data sources include data from a physiological sensor and a caregiver data store, a connector and source are paired, and each paired connector converts source data between a first format and a second format different from the first format;
a rules engine and a medical rule associated with a medical protocol; and
a core transaction processing system configured to apply the medical rule to the data in the second format to implement a compliance rules check of the medical protocol.

22. The system of 21, wherein the application of the medical rule confirms patient compliance with the medical protocol.

23. The system of 21, wherein the application of the medical rule confirms caregiver compliance with the medical protocol.

24. The system of 21, wherein the application of the medical rule generates health status data.

25. The system of claim 24, wherein the health status data is used for real-time monitoring of the patient.

26. The system of 21, wherein the application of the medical rule generates a mental health profile of the patient.

27. The system of claim 21, wherein the data sources further comprise one or more of a body area network sensor, an EHR, an EMR, an HIS record, a public personal profile, a restricted personal profile, geo-location data, or route data.

28. The system of claim 21, wherein the connector converts data from the second format to the first format and the system generates an external actionable event.

29. The system of claim 21, further comprising a data integrator to collate data from at least two data sources.

30. The system of claim 21, further comprising a display to view the data from the data sources in the second format.

31. The system of claim 21, wherein the medical protocol comprises one or more of a quality of service protocol, a mental health check protocol, a post procedure complication monitoring protocol, a diabetes monitoring protocol, a vital sign monitoring protocol, a fraud prevention protocol, a delivery of prescribed services protocol, or a combination thereof.
